(19) United States
(12) Patent Application Publication
(10) Pub. No.: US 2020/0087604 A1
(43) Pub. Date: Mar. 19, 2020

(54) LARGE SCALE CELL GROWING DEVICES AND SYSTEMS

(71) Applicants: Stephen John Pearson, Antioch, IL (US); Justin Zdeb, Mount Dora, FL (US)

(72) Inventors: Stephen John Pearson, Antoch, IL (US); Justin Zdeb, Mount Dora, FL (US)

(21) Appl. No.: 16/567,777

(22) Filed: Sep. 11, 2019

Related U.S. Application Data
(60) Provisional application No. 62/765,793, filed on Sep. 14, 2018.
(60) Provisional application No. 62/921,945, filed on Jul. 16, 2019.

(51) Int. Cl. C12M 1/24; C12M 3/00; C12M 1/00; C12N 5/00; C12N 5/07; C12M 1/34

(52) U.S. Cl. CPC C12M 23/08; C12M 23/44; C12M 23/58; C12N 5/0018; C12M 41/40; C12M 29/18; C12M 41/30; C12M 41/16; C12N 5/06

(57) ABSTRACT

A cell-growing system may include a cell-growing container, a pump for controlling the pressure of the chamber of the container, a filter for maintaining the chamber sterilized, a flow control regulator for controlling the substance supplied to the cell-growing container. The cell-growing container may include an internal structure that takes the form of a reticulated structure to increase the surface area for growing cells. The cell-growing system may be used to perform cycles of cell expansion and harvest. The cell-growing system may be maintained as a closed and sterilized system through the cycles. In harvesting, a first subset of the cells can be disassociated from the cell culture while a second subset remains. The disassociated cells may be discharged to a collection container while maintaining the system closed to an external environment. The remaining cells may be used as seeds to proliferate more cells in the next cycle.

### CROSS REFERENCE TO RELATED APPLICATIONS

The present application claims the benefit of U.S. Provisional Patent Application No. 62/765,793, filed on Sep. 14, 2018, and Provisional Patent Application No. 62/921,945, filed on Jul. 16, 2019, both of which are hereby incorporated by reference in their entirety.

### BACKGROUND

To grow biological cells in an artificial environment, it is usually desirable to keep unwanted organisms and contaminants out of the environment. Conventionally, in biological laboratories, manipulation of a cell-growing medium is typically performed under a laminar flow hood using pipettes to add or remove components like nutrients or waste products from the cell culture. Conventionally, cell culture operations usually use styrene flasks to grow cells. Cells in a solution are added onto the surface of a flask, manually spread on top of the coating of the flask, and are allowed to drift and adhere to the bottom surface of the flask. If the conditions are right, the cells will proliferate and spread out on the entire surface until the surface is saturated. To further expand the number of cells, agents such as trypsin are introduced to break down the adherent protein and release the adherent cells back into a solution. The solution will be transferred to additional flasks to repeat the manual spreading and proliferation process to double the number of cells. The process can further be repeated until the desired number of cells are achieved. However, these practices are often prone to contamination, especially in transferring cells between flasks.

Conventional standard operating procedures use an open system where cells are possibly exposed to contamination during the manual process of opening flasks to remove feeding media over a period of time. Those systems and processes fail to provide stable and sterilized growth conditions for cells that are close to the native environment, thus often causing stress on the cells during the growth process. These shortcomings could be particularly problematic when growing stem cells. When conditions are not ideal, the stress on the stem cells could cause the cells to slowly differentiate and try to adapt with each division to the foreign environment.

### SUMMARY

The present disclosure relates to a cell-growing system and, more particularly, to a large-scale cell-growing system that can grow cells in a closed and sterilized environment.

Embodiments disclosed herein relate to a system for growing biological cells in a closed environment. The system may include a cell-growing container for growing biological cells. The cell-growing container includes a body and a port. The body defines the chamber of the cell-growing container. The system may include a pump in communication with the cell-growing container through a tube connected to the port. The pump may regulate the pressure of the chamber. The system may also include a filter located between the cell-growing container and the pump. The filter prevents contamination of the chamber. The system may also include additional components such as a flow control regulator and a collection bag.

The system provides an environment for cells to proliferate in a closed and sterilized fashion. The system allows cells to be repeatedly grown and harvested in cycles while maintaining the cell-growing environment closed and sterilized. The cycles may be a series of a cell expansion phase and a harvest phase. In a cell expansion phase, the cells may be grown in an environment with regulated pressure and temperature. Also, nutrients may be continuously fed to the chamber of the cell-growing container in a controlled manner. In a harvest phase, a subset of the cells may be collected from the chamber while another subset of the cells may remain in the chamber as the seeds for the next cell expansion phase.

Embodiments disclosed herein also relate to a container for growing biological cells. The cell-growing container may include a body defining a chamber and a reticulated structure located in the chamber. The reticulated structure may be defined by a frame. The frame defines the volume of the reticulated structure and creating a plurality of spaces on and inside the volume. The frame may include a plurality of cell-growing subunits. Each cell-growing subunit may include a first surface formed of a material suitable for growing biological cells and a second surface also formed of the material but oriented differently from the first surface. The cell-growing container may also include a port. The port allows substance exchange between the chamber and an external source.

Embodiments disclosed herein further relate to a method of growing cells. A user may apply biological cells on one or more cell-growing surfaces of a cell-growing container. The user may close the cell-growing container from an external environment. The user may allow the biological cells to proliferate in the cell-growing container. The user may apply a force external to the cell-growing container to disassociate a first subset of biological cells from the cell-growing surfaces. A second subset of biological cells may remain on the cell-growing surfaces. The user may connect the cell-growing container to a collection container. The cell-growing container may remain closed from the external environment when connected to the collection container. The user may further cause the discharge of the first subset of biological cells to the collection container.

### BRIEF DESCRIPTION OF THE DRAWINGS

Figure (FIG.) 1 is a perspective view of an example cell-growing system, in accordance with an embodiment.

FIG. 2 is a perspective view of an example cell-growing container that has switches controlling the opening and closing of its ports, in accordance with an embodiment.

FIG. 3 is a perspective view of an example cell-growing container, in accordance with an embodiment.

FIG. 4 is an exploded view of the cell-growing container shown in FIG. 3.

FIG. 5 is a cross-section view of the cell-growing container shown in FIG. 3.

FIG. 6 is a cross-section view of a cell-growing container that has an internal tube, in accordance with an embodiment.

FIG. 7 is a conceptual diagram illustrating an example cell-growing structure, in accordance with an embodiment.

FIG. 8 is a conceptual diagram of different possible cross-section shapes of a cell-growing subunit, in accordance with various embodiments.

FIG. 9 are two side views of an example cell-growing container, in accordance with an embodiment.

FIG. 10 shows an internal pipette for a cell-growing container, in accordance with an embodiment.

FIG. 11 is a conceptual diagram illustrating an example cell-growing container, in accordance with an embodiment.

FIG. 12A is a perspective view of an example cell-growing container, in accordance with an embodiment.

FIG. 12B is a cross-section perspective view of the example cell-growing container, in accordance with an embodiment.

FIG. 13 is a flowchart depicting an example cell-growing process, in accordance with an embodiment.

The figures depict, and the detail description describes, various non-limiting embodiments for purposes of illustration only.

### DETAILED DESCRIPTION

Reference will now be made in detail to embodiments, examples of which are illustrated in the accompanying drawings. In the following detailed description, numerous specific details are set forth in order to provide a thorough understanding of the various described embodiments. However, the described embodiments may be practiced without these specific details. In other instances, well-known methods, procedures, and components have not been described in detail so as not to unnecessarily obscure aspects of the embodiments.

### Example System

Figure (FIG.) 1 is a perspective view of an example cell-growing system 100, in accordance with an embodiment. The cell-growing system 100 may include a cell-growing container 110 for growing biological cells, a pump 120 for controlling the pressure of the internal space of the cell-growing container 110, a filter 130 for maintaining the internal space sterilized, a flow control regulator 140 for controlling the substances supplied to the cell-growing container 110, and a collection container 150 for receiving media and outputs from the cell-growing container 110. The cell-growing system 100 may include fewer or additional components not shown in FIG. 1 and may also include different components.

The biological cells may be stem cells, tissue cells, other human cells, mammalian cells, other organisms' cells, unicellular organisms, etc. The cells may be adherent cells or suspension cells. Adherent cells are cells that grow on a surface such as a tissue culture or a coating. Suspension cells are cells that may freely suspend and grow in a culture medium solution. The cell-growing system 100 is suitable for growing both adherent cells and suspension cells. In one case, the biological cells used for the system 100 may be stem cells such as mesenchymal stem cells, but the system 100 is also suitable for proliferating other types of biological cells.

The cell-growing container 110 includes a body 112 that defines a chamber 114 for growing biological cells. The chamber provides an internal space for growing cells. In various embodiments and depending on the configuration and design of the cell-growing container 110, the cell-growing container 110 may sometimes also be referred to as a cell culture flask, a cell culture partition, a cell culture dish, a vial, a bioreactor and/or a centrifuge tube. The body 112 may be formed of any suitable materials, natural or synthetic, such as glass, hard or soft plastic, or other suitable polymers. The body 112 or part of it may be transparent to allow the cells to be observed under a microscope. The chamber 114 of the cell-growing container 110 may include an internal structure or internal components (not shown in FIG. 1) that provide additional surface area for biological cells to grow. For example, the internal structure may increase the available surface area by 10, 100, 1000, or 10,000 times. The details of some example internal structure will be further discussed with reference to other figures. The cell-growing container 110 provides an environment that is suitable for the expansion of both adherent cells and suspension cells.

The cell-growing container 110 may form a closed chamber 114, which may be isolated from the external environment 160. The cell-growing container 110 may include one or more ports that permit a limited and controlled exchange of substances between the chamber 114 and a source or a destination. For example, in the example embodiment shown in FIG. 1, the cell-growing container 110 may include a first port 116 and a second port 118. The first port 116 may be connected to the pump 120 through a first tube 122. The filter 130 may be located at an intermediate point of the first tube 122 to prevent external unwanted substances and contaminants from entering the closed chamber 114. Through a second tube 152, the second port 118 may be connected to the collection container 150 that is used to receive media, waste, and/or proliferated cells from the cell-growing container 110. During a cell expansion phase and a harvest phase, the cell-growing container 110 may remain closed and isolated from the external environment 160.

The tubes may include one or more Y-connector 124 to allow additional sources of substances to be added to the chamber 114 and to allow an initial application of cells. In FIG. 1, a Y-connector 124 is shown along the first tube 122, but the second tube 152 may also include a Y-connector. In growing cells such as stem cells, a user may initially add the cells to chamber 114 through a syringe that collects the cells from a sterile vial. The syringe's needle draws the cells from the sterile vial and ejects the cells to Y-connector 124 into the chamber 114. As such, the injection of the cells can be done in a sterilized manner when the chamber 114 remains closed from the external environment 160.

In this context, that the cell-growing container 110 is closed from the external environment 160 does not necessarily require the cell-growing container 110 be completely sealed. The cell-growing container 110 may still be in communication with other components such as a nutrient source, the pump 120, etc. However, when the cell-growing container 110 is closed from the external environment 160, either that the cell-growing container 110 and a component connected to the cell-growing container 110 together form a closed system or that, if a component is not closed, substances provided to the cell-growing container 110 through a communication channel (e.g., the channel through first port 116 or second port 118) connected to the component is filtered and sterilized. The filtering and sterilization may be performed by an assembly of filter components 130.

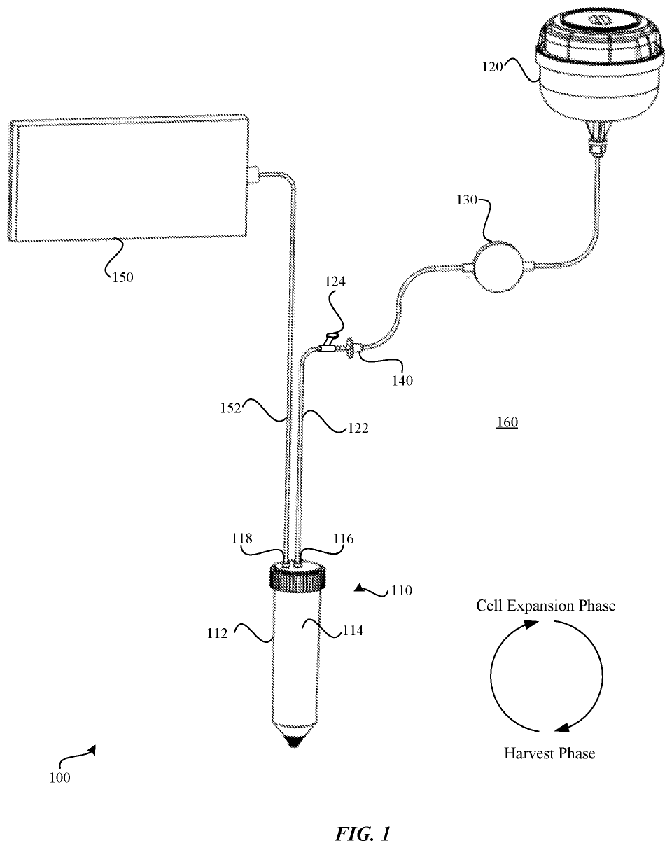

The cell-growing container 110 may be removably coupled to different components. For example, FIG. 2 is a perspective view of an example cell-growing container 110 that has switches controlling the opening and sealing of its ports, in accordance with an embodiment. The first port 116 and second port 118 may each include a short segment of tubing that has a switch 202 to open or seal the port. The switch 202 may be connected to a tube 204, which in turn provides a pathway for the chamber 114 to communicate with other components. The end of the tube 204 has an adaptor 206 for a connection with another component. The system 100 may switch components that are in communication with the cell-growing container 110. In switching the components, the switch 202 may be closed first before the adaptor 206 is detached from the adaptor of another component, thereby maintaining the isolation of closed chamber 114 from the external environment 116. The type of components that are in communication with the cell-growing container 110 may change during different phases of cell expansion and harvest. For example, in one case, the cell-growing container 110 may be completely sealed with both ports 116 and 118 closed from any components during a cell expansion period. In another case, during a cell expansion period, the cell-growing container 110 may be connected to the pump 120 and various substance sources but is maintained closed and sterilized from the external environment 160. The cell-growing container 110 may also be sealed and removed from the system 100 for various operations, such as a centrifugal operation, before the cell-growing container 110 is re-connected to other components of the system 100. In some cases, multiple collection containers 150 may be connected to the cell-growing container 110 from time to time. For example, a first collection container 150 may be connected to receive a waste discharge of the cell-growing container 110 and a second collection container 150 may subsequently be connected to receive proliferated cells.

The system provides a closed and sterilized environment in the chamber 114 for cells to be repeatedly grown in cycles. The cycles may be a series of a cell expansion phase and a harvest phase. In a cell expansion phase, pressure and temperature may be regulated and nutrients may be continuously fed to the chamber 114 of the cell-growing container 110 in a controlled manner. In a harvest phase, a first subset of the cells may be collected from the chamber 114 while a second subset of the cells may remain in the chamber 114 as the seeds for the cell expansion phase in the next cycle.

The cell-growing container 110 may serve as a batch or semi-batch bioreactor to allow multiple cycles of cell expansion and harvest to produce a large number of cells under a single cell-growing container 110. For example, after an initial number of cells is applied to the cell culture of the cell-growing container 110, the biological cells proliferate in a cell expansion phase. Substances such as nutrients and gas may be continuously supplied during the cell expansion phase. After the cells multiply, the cell-growing container 110 may be temporarily removed from the system 100 for an operation to disassociate a subset of the proliferated cells from the cell culture. The disassociation operation may include a physical operation and/or a chemical operation to release the subset of cells. For example, a force (e.g., a centrifugal force, the gravitational force, a sudden acceleration) may be applied to remove some cells from the cell culture. Additionally, or alternatively, an agent, such as trypsin or another suitable enzyme, may be added to the chamber 114 of the cell-growing container 110 to release some of the cells. After the disassociation operation, the cell-growing container 110 may be connected to the collection container 150 and the subset of cells may be flushed or otherwise discharged to the collection container 150. Another subset of cells may remain in the chamber 114. An operator may repeat the cell expansion and harvest process in a second batch. Throughout the cycles, the system 100 is closed from the external environment 160 to prevent the chamber 114 from being contaminated.

The pump 120 may serve different purposes in different phases of cell expansion and harvest. For example, during a cell expansion phase, the pump 120 may be in communication with the cell-growing container 110 to regulate the pressure inside the chamber 114. The pump 120 may pressurize the chamber 114 to reduce the chance of foreign unwanted objects (e.g., contaminants and microorganisms) from entering the chamber 114. In one embodiment, the pump 120 may maintain the pressure inside the chamber 114 to a value that is higher than the atmospheric pressure. In some cases, the pump 120 may also regulate the pressure of the chamber 114 to prevent harmful pressure buildup inside the chamber 114. The internal pressure of the chamber 114 may also be regulated and maintained with a pressure relief valve (not shown) that is connected to one of the tubes, by adding gas through a sterile filter 130 into the chamber 114, by pressure generated as a result of reactions inside the device, by the pressure controlled by the pressure of the gas introduced, and/or the volume of the gas or liquid pumped into the chamber 114. Solid parts added to the chamber 114 may also affect the internal pressure.

The pump 120 may also be used to regulate the substances supplied to the cell-growing container 110 during a cell expansion phase. For example, the pump 120 may work with the filter 130 and the flow control regulator 150 to regulate various substance sources (not shown in FIG. 1) that may be connected to the cell-growing container 110. The substances may be supplied to the chamber 114 through the first rube 122, which may also be referred to as a feeding tube.

The filter 130 may prevent unwanted external substances from entering the cell-growing environment of the chamber 114. The filter may be an inline sterile filter. The filter 130 may be an assembly that includes one or more types of filtering components. For example, the filter 130 may include a mechanical filter that includes one or more porous layers to filter substances of different sizes. A filter paper is an example of a mechanical filter. In one embodiment, a 0.22-micron filter or a filter with similar porous size may be used to maintain sterility of the chamber 114. The filter 130 may also include other types of filtering components such as a chemical filter that is formed of a suitable chemical agent such as activated carbon. The filter 130 may further include a sterilizer such as an ultraviolet sterilizer.

The flow control regulator 140 may be a flow control valve that cooperates with the pump 120 to regulate the pressure and substance supplies of the cell-growing container 110. The flow control regulator 140 may restrict the passage of the tubes, for example, by reducing the cross section of the passage by half. One or more flow control regulators 140 may be used and may be connected to different substance sources. In one embodiment, the substance sources may include an oxygen source and a carbon dioxide source. Additionally, or alternatively, the substance sources may also include various nutrient sources and cell medium sources. The flow control regulators 140 may regulate the amounts of substances supplied to the chamber 114. The flow control regulators 140 may be connected to and controlled by a computer (not shown in FIG. 1) to control the amount of substances supplied to the chamber 114. The flow control regulators 140 may regulate the amount of oxygen and carbon dioxide in the chamber 114 to mimic a native biological environment of the biological cells being grown (e.g., a human organ's environment at which the biological cells naturally grow). For some native biological environments, lower than the atmospheric level of oxygen is observed, the flow control regulators 140 may limit the supply of oxygen to the chamber 114.

The pump 120 may also be used to discharge substances and cells from the cell-growing container 110 to a collection container 150. For example, in a harvest phase, a subset of the proliferated cells may be disassociated from the cell culture. The cell-growing container 110 is connected to the collection container 150 through a second tube 152, which may be referred to as a discharge tube. The pump 120 may increase the pressure of the chamber 114 to force the cells out of the cell-growing container 110 to the collection container 150.

The collection container 150 may be in any suitable size and shape. The collection container 150 may be any suitable receptacle such as a flask, a vial, a collection bag, a tube such as a centrifugal tube and an Eppendorf tube, a bottle, and another cell-growing container 110. The collection container 150 may include a small tube or a cracking pressure valve. In a harvest phase, the collection container 150 may be connected to the cell-growing container 110 and the entire system may be closed to the outside environment 160 so that the harvesting may be performed in a closed and sterilized manner. The cells in the cell-growing container 110 may be discharged to the collection container 150, such as through the pump 120 that applies pressure to push the cells to the collection container 150. After the cells are transferred, the connection between the cell-growing container 110 and the collection container 150 may be closed. For example, the cell-growing container 110 may have a switch 202 that is shown in FIG. 2 that can be sealed. The collection container 150 may subsequently be detached from the system 100. Other types of collection container 150 may also be connected to the cell-growing container 110 in other phases. During a cell expansion phase, the waste collection container 150 may be used to collect wastes continuously from the cell-growing container 110. The waste collection container 150 may also receive released gas to help regulate the pressure in chamber 114.

Other components not shown in FIG. 1 may also be presented in the system 100. For example, throughout a cell expansion phase, the cells may proliferate in a temperature-controlled environment. The cell-growing container 110 may be warmed by an incubator.

### First Example Cell-Growing Container

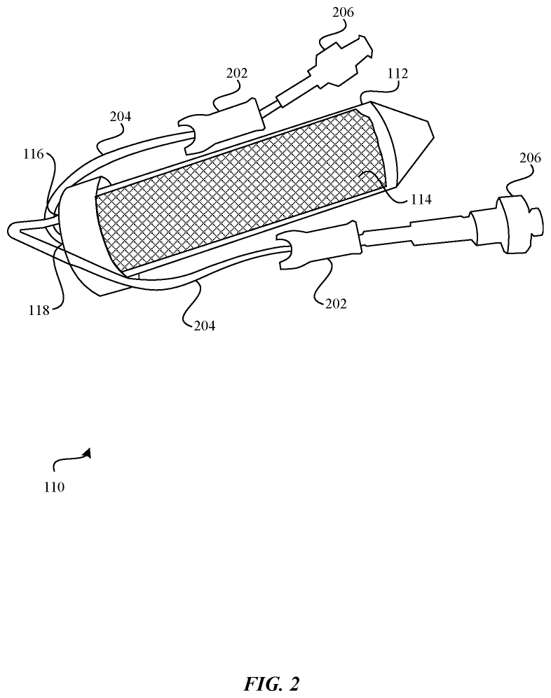

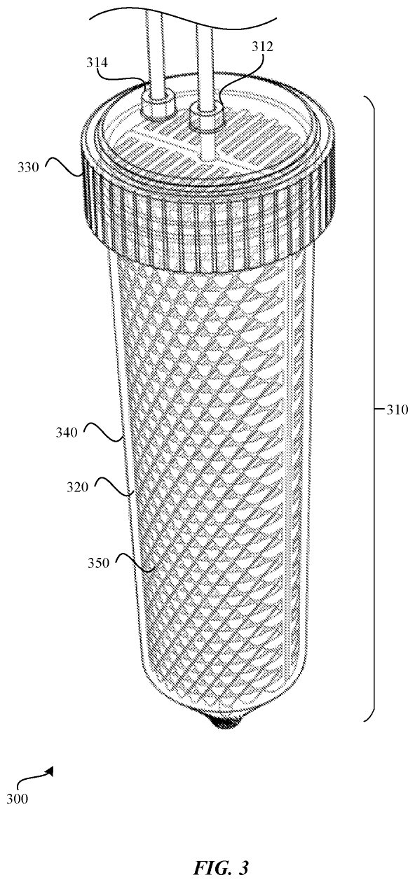

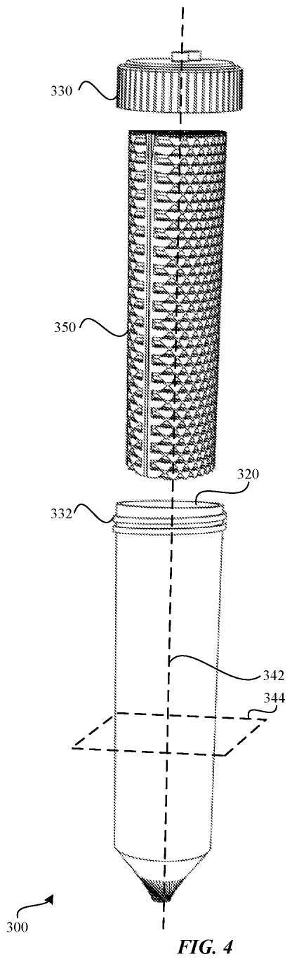

FIGS. 3, 4, and 5 are various views of an example cell-growing container 300, in accordance with an embodiment. The cell-growing container 300 is an example of the cell-growing container 110 that may be used in system 100 shown in FIG. 1. FIG. 3 is a perspective view of the cell-growing container 300 with the outer structure made transparent to show the interior structure. FIG. 4 is an exploded view of the cell-growing container 300 showing an interior cell-growing structure. FIG. 5 is a cross-section view of the cell-growing container 300 showing an example arrangement of tubing position.

With reference to FIGS. 3, 4, and 5, the cell-growing container 300 includes a body 310 that defines a chamber 320 for carrying cells and providing an environment for cell proliferation. The body 310 may include multiple ports, such as a first port 312 and a second port 314, that permit substance exchange between the chamber 320 and another component, such as a pump, a collection container, etc. that is shown in FIG. 1. The body 310 may include a cap 330 and a receptacle 340. The body 310 may carry an internal structure 350 that may serve as the substrate for growing a large number of cells. The internal structure 350 may be referred to as a cell-growing structure 350. The cell-growing container 300 may include fewer or additional components that are not shown in various figures. The cell-growing container 300 may also include different components.

The body 310 may be a unitary body or may be formed of multiple parts. For example, in the example shown in FIG. 3, the body 310 includes the cap 330 that is removably engaged with the receptacle 340 to cooperatively form the chamber 320. Even though the cap 330 is removable, the cap 330 may not necessarily need to be completely detached from the receptacle 340. For example, for an embodiment of the body 310 that resembles an Eppendorf tube (not shown in figures), there can be a hinge that connects the cap 330 to the receptacle 340. The cap 330 may be engaged with the receptacle 340 via any suitable structure. For example, both the cap 330 and the receptacle 340 may include screw threads 332 that are complementary to each other. Other engagement methods such as frictional fit, locks, adhesive, etc. may also be used. The body 310 may also include additional sealing structure such as an O-ring, a coating, a sealant, etc. at the interface between the cap 330 and the receptacle 340 to provide sealing for the chamber 320. The cap 330 provides a larger opening for the receptacle 340 for the initial application of cells. The interface between the cap 330 and the receptacle 340 may subsequently be sealed so that substance exchange between the chamber 320 and a substance source or destination is mainly achieved through the first and second ports 312 and 314. While the first and second ports 312 and 314 are shown to be located at the cap 330, one or more of the ports may be located at different parts of the body 310.

The body 310 may be of any suitable size and any suitable shape. In one embodiment, the body 310 may be sized and shaped to be insertable to a centrifuge (not shown in the figures). A centrifuge in this context may include any suitable revolving machines such as a conventional centrifuge, a blender, etc. The centrifuge may be a benchtop centrifuge. For example, common benchtop centrifuges may be configured into to swing-bucket centrifuges and fixed-angle centrifuges. Each type of centrifuges may include one or more holders, such as slots or arms, for holding containers. In various embodiments, a body 310 may be sized and shaped in accordance with the holders of a centrifuge that is compatible with the cell-growing container 300. In this context, the body 310 being sized and shaped to be insertable to a centrifuge does not necessarily mean that the body 310 has the precise size and shape that are complementary to, for example, a slot of the centrifuge. Instead, the body 310 may have the size and shape to be held securely by the centrifuge during a centrifuge operation. For example, if the holder of the centrifuge is a ring or a slot, the receptacle 340 may have a first circumference that is smaller than the holder's internal circumference while the cap 330 may have a second circumference that is larger than the holder's internal circumference. Other designs of size and shape of the body 310 may also be possible for the centrifuge to securely hold the cell-growing container 300 during a centrifuge operation.

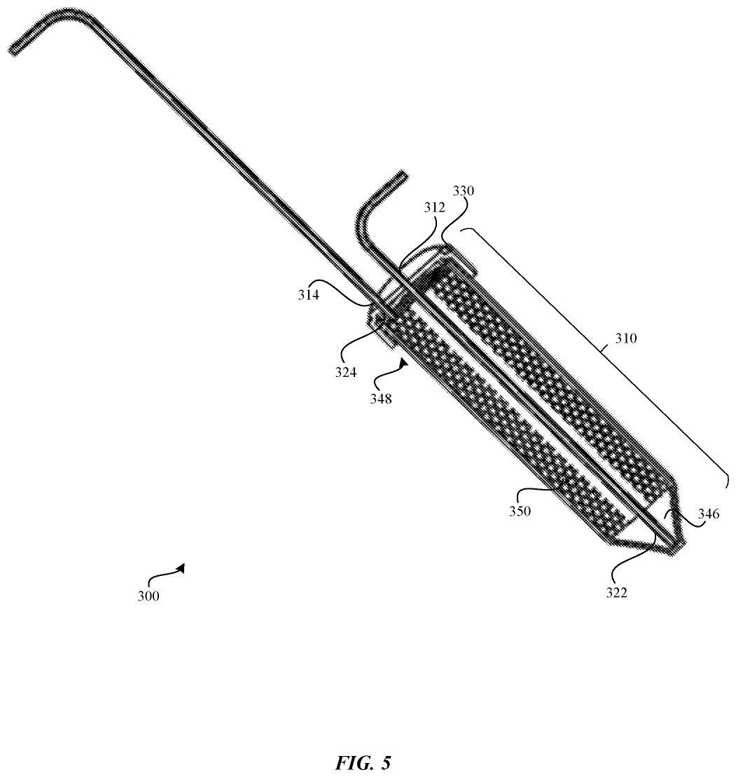

A cell-growing structure 350 is carried by the body 310 in the chamber 320 of the cell-growing container 300. In various embodiments, the body 310 may carry the cell-growing structure 350 in different ways. For example, the cell-growing structure 350 may be permanently mounted to the body 310 through one or more mechanical connections. In another example, the cell-growing structure 350 may be removably inserted into the chamber 320. In such a case, initially, a user may initially apply the cells on the outer surface of the cell-growing structure 350 and insert the cell-growing structure 350 into the receptacle 340 before the chamber 320 is closed by the cap 330. The cell-growing structure 350 may serve as the substrate by providing surfaces for cells to grow and may have a configuration that permits cells to proliferate at a large scale. The cell-growing structure 350 may take the form of a reticulated structure to increase the surface areas for growing cells. Possible configurations and design of the cell-growing structure 350 will be discussed in further details below with reference to FIGS. 6, 7, 12A and 12B.

At least part of the surface of the cell-growing structure 350 may be formed of materials that are suitable for growing biological cells. For example, the cell-growing structure 350 may include multiple layers. The cell-growing structure 350 may include an interior skeleton that is formed of a more rigid material such as polystyrene, fiberglass, or another suitable polymer. The exterior of the cell-growing structure 350 may be treated with one or more layers of coatings that provide the suitable medium and adhesion for biological cells to grow. The coatings may be formed of any suitable materials. For example, the materials may be hydrophilic to provide better adhesion of the biological cells. In one embodiment, the hydrophilicity of the coating material is higher than the surface adhesion of biological cells. Example coating materials may include suitable chemicals, amino acids, and proteins such as collagen, retronectin, poly-lysine, streptavidin, antibodies, and polyethyleneimine. The materials may also be referred to as cell attachment materials.

To provide sufficient area for cell growth, the cell-growing structure 350 may occupy the bulk of the chamber 320. For example, the volume of the cell-growing structure 350 may be at least fifty percent of the volume of the chamber 320. In one embodiment, the receptacle 340 may have an elongated shape that can be characterized as having a longitudinal axis 342 and a cross-section 344. The cell-growing structure 350 may also be elongated along the longitudinal axis and have a cross-section that is at least sixty percent of the cross-section of the receptacle 340.

While the cell-growing structure 350 is shown as having a reticulated structure, in some embodiments the cell-growing structure 350 may also be a simple cylinder that provides external surfaces for cell growth.

Referring to FIG. 5, in some embodiments, the ports 312 and 314 may be coupled to tubing that extends to different locations of the chamber 320. The chamber 320 may be divided into different regions based on the position of the cell-growing structure 350. For example, the receptacle 340 may include a closed end 346 and an open end 348 that is configured to be engaged with the cap 330. The port 312 may be connected to a first internal tube 322 and the second port 314 may be connected to a second internal tube 324. The first internal tube 322 may extend or penetrate through the cell-growing structure 350 to the closed end 346 while the second internal tube 324 may only extend to the open end 348. In one embodiment, the first internal tube 322 is in communication with a pump 120 while the second internal tube 324 is in communication with a collection container 150. In a cell expansion phase, the first internal tube 322 may serve as an input feeding tube to provide nutrients to the chamber 320 and the second internal tube 324 may serve as an exit tube to collect old media. In a harvest phase, an external force may be applied to the cell-growing container 300 to disassociate a subset of cells. As will be discussed in further details, the disassociated cells will largely accumulate at the closed end 346. The first internal tube 322, when extends to the closed end 346, may blow air that is generated by the pump 120 to flush the cells to the collection container 150 via the second internal tube 324.

The internal tubes may also be pipettes. For example, a pipette may have a relatively rigid tip. A flexible tube may be used to connect the tip and a port. In some embodiments, a cell-growing container may include multiple ports and multiple pipettes. Some of the pipettes have fixed positions while others are movable. The tips that are movable may be used for pointing to a particular position and may also be used for mechanical action such as scraping.

FIG. 6 is a cross-section view of a possible arrangement of a cell-growing container 300, in accordance with an embodiment. In addition to, or alternative to, the cell-growing structure 350, the cell-growing container 300 may include a U-shape tube 360 for the preservation of suspension cells. The tube 360 may be any suitable container and may take a shape other than the U-shape. In this arrangement, the cell-growing container 300 may be turned into a container for growing suspension cells. In growing suspension cells, the chamber 320 may be filled with a suitable solution that serves as the cell-growing medium. In a harvest phase, the solution along with the proliferated cells may largely be drained to a collection container 150 by the pump 120. The U-shape tube 360 preserves a small number of suspension cells inside the chamber 320 for the next cycle of cell expansion. In growing suspension cells, the cell-growing structure 350 may sometimes be removed from the chamber 320.

In some embodiments, the cell-growing structure 350 is replaced by or complemented by a plurality of loose cell-growing units such as beads, spheres, or ribs with regular or irregular shapes. The loose cell-growing units have surfaces that are coated with materials suitable for growing cells. The cell-growing units may serve a similar role to the cell-growing structure 350 by increasing the overall surface area for growing cells.

### Example Cell-Growing Structure

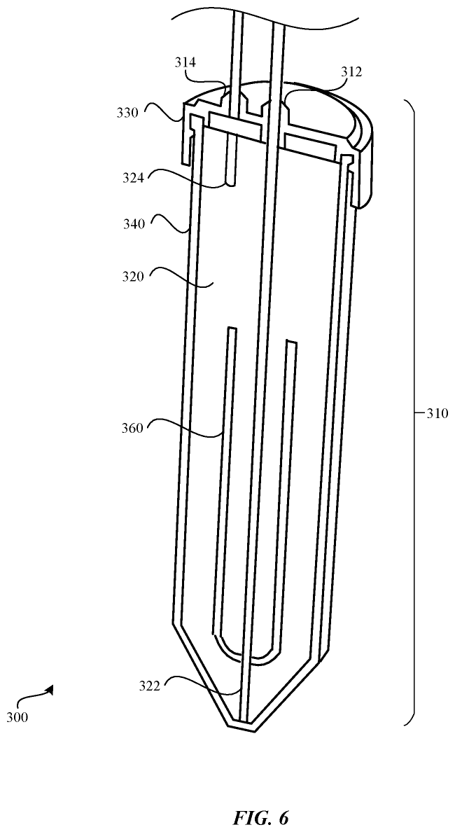

FIG. 7 is a conceptual diagram illustrating an example cell-growing structure 700, in accordance with an embodiment. The cell-growing structure 700 may be an example of a cell-growing structure 350 located in the cell-growing container 300. FIG. 7 illustrates one possible design and configuration of a cell-growing structure. Other designs and configurations may also be possible.

The cell-growing structure 700 may be a reticulated structure that is located in the chamber of a cell-growing container 300. The cell-growing structure 700 includes a frame 710, which is the mechanical structure that defines the volume and shape of the cell-growing structure 700. The frame 710 also creates a plurality of spaces 720 (e.g., cavities, best shown in inset 708) that are present both on the surface of the volume of the cell-growing structure 700 and within the volume. The frame 710 may include a plurality of cell-growing subunits 730. The large number of spaces 720 around the cell-growing subunits 730 provides an increased surface area for growing biological cells. The surface of the cell-growing subunits 730 may be formed of a material suitable for growing biological cells. In one case, the total surface area of the cell-growing structure 700 is at least ten times larger than the outer surface area of the cell-growing structure (e.g., the surface area of the cylinder if the structure was not reticulated). In other cases, the total surface area of the cell-growing structure 700 can be at least a hundred times or a thousand times larger than the outer surface area. Likewise, the total surface area of the cell-growing structure 700 can be at least ten times, a hundred times, or even a thousand times larger than the inner surface area of the body of the cell-growing container 300.

The body of the cell-growing structure may have any shape and may not necessarily have the regular circular cylindrical volume as shown in FIG. 7. For the particular embodiment illustrated in FIG. 7, the body of the cell-growing structure 700 may be characterized as having a longitudinal axis 702 and a cross-section 704. The frame 710 includes a plurality of cell-growing subunits 730 that may be arranged in multiple layers 740 along the longitudinal axis 702. The plurality of cell-growing units 730 may form a matrix pattern 750. In the arrangement shown in the embodiment of FIG. 7, the matrix pattern 750 may include layers 740 that are regularly arranged and have roughly the same number of cell-growing subunits 730 for each layer 740. Each layer 740 may be parallel to other layers 740. In another embodiment not shown in FIG. 7, the cell-growing subunits 730 may be arranged in a staggered manner so that the matrix pattern 750 may not have well-defined layers. In yet another embodiment not shown in FIG. 7, the cell-growing subunits 730 may be arranged randomly so that the matrix pattern 750 may have an irregular pattern. While in the embodiment shown in FIG. 7 each cell-growing subunit 730 may has a relatively well-defined boundary that separates the cell-growing subunit from another one (e.g., the heart shape can be well-defined boundary), in other embodiments not shown in the drawings the cell-growing subunits 730 can be continuous and do not necessarily need to have well-defined boundaries.

Referring to the inset 706, a cross-sectional view of the frame 710 that is generally perpendicular to the longitudinal axis 702 is shown. Each cell-growing subunit 730 may take the form of an elongated beam that is cantilevered or otherwise extended from the middle of the entire frame 710. In other embodiments, the cell-growing subunits 730 may take other shapes and may not necessarily be extended from a certain part of the frame 710 or may not be in a beam shape. Within a layer 740, the cell-growing subunits 730 may be arranged in parallel to each other, as shown in inset 706. In another embodiment not shown, the cell-growing subunits 730 within a layer 740 may be arranged radially from the center of the frame 710. In yet another embodiment not shown, the cell-growing subunits 730 within a layer 740 may be arranged randomly and extend at different random directions.

Referring to the inset 708, an enlarged view of an example matrix pattern 750 is shown. The cross-section of each cell-growing subunit 730 may have the same shape or a different shape. For example, the shape may be a heart shape. The shape may also be a circle, any polygon, such as a triangle, a square, a trapezoid, a hexagon, or any suitable shape, regular or irregular, symmetrical or not. Each cell-growing subunit 730 may also have the same size or a different size.

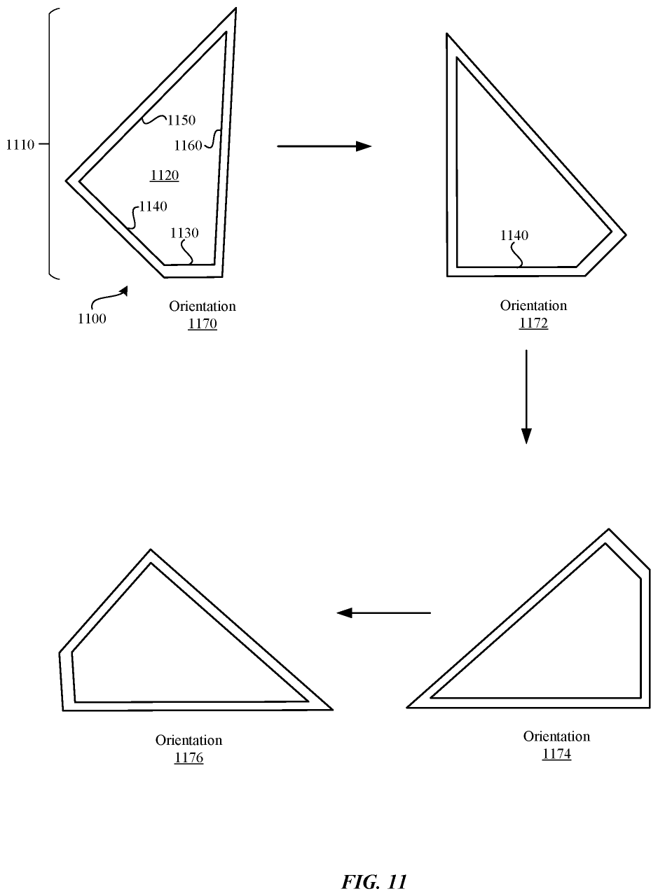

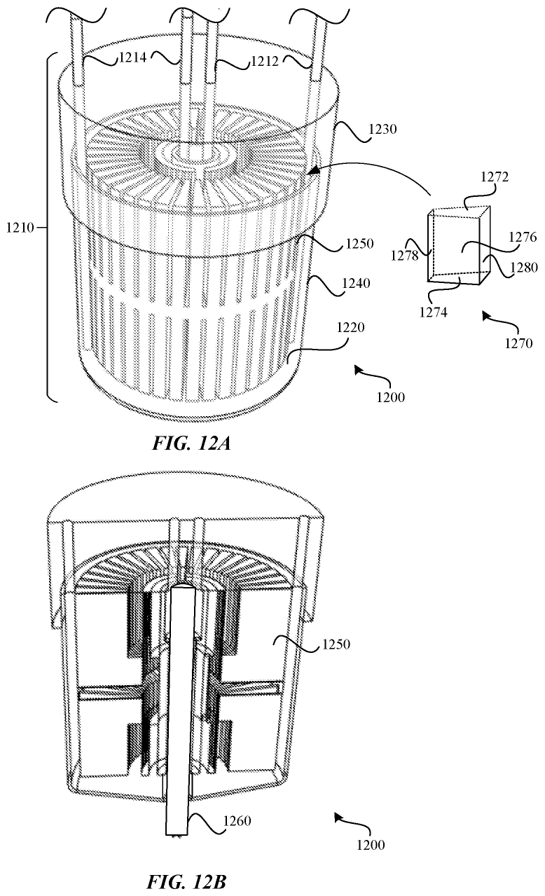

The cell-growing subunits 730 are spaced apart to create a plurality of spaces 720 among the cell-growing subunits 730. The design and configuration of the cell-growing subunits 730 create a reticulated structure for the cell-growing structure 700. A reticulated structure is a surface-increasing structure and may define spaces to increase the total surface of the structure. The total surface area of a reticulated structure is larger than the outer surface area of the structure. For example, the total surface area of the reticulated structure 700 is larger than the outer surface area of the structure 700 (e.g., the outer surface area in this case is the surface area of the cylinder). A reticulated structure is not necessarily associated with a netted or meshed structure. Depending on the configuration and the shape of the cell-growing subunits 730, the reticulated structure may include and may be referred to as a matrix structure, a porous structure, a honeycomb-like structure (even though the shape may not necessarily be hexagonal), a foamed structure, a netted structure, a meshed structure, a fanned structure and/or a corrugated structure. A reticulated structure is not limited to the shape and configuration shown in FIG. 7. For example, a fanned structured shown in FIGS. 12A and 12B is another example of a reticulated structure. The spaces 720 may be presented both on the outer surface (e.g., the cylindrical circumferential surface) of the volume of the cell-growing structure 700 and inside the volume. The spaces 720 create cell-growing surfaces for the cell-growing subunits 730 on the surfaces of the frame 710. The cell-growing subunits 730 may also be hollow (e.g., having a hollow heart shape) to further increase the surface area suitable for growing cells.

A cell-growing subunit 730 may include a plurality of surfaces that are formed of a material suitable for growing biological cells and that are oriented differently. For example, an example cell growing subunit 730 may include a first surface 734 and a second surface 736 that is oriented differently from the first surface. The cell-growing subunits 730 may include additional surfaces that are oriented differently. A surface may be straight or curved. In one case, the first and second surfaces may be generally orthogonal (within 5 degrees plus or minus of 90 degrees) to each other. In another case, the first and second surfaces may face opposite directions. The different orientations of cell-growing surfaces allow a subset of cells that are grown on the cell-growing structure 700 to be disassociated from the structure and allow another subset of cells to remain on the cell-growing structure 700. For example, when a centrifugal force 760 that is generally perpendicular to the rotational axis 770 is applied through a centrifuge, at least majority of the cells on the first cell-growing surface 734 will remain on the cell-growing subunit 730.

The cell-growing subunit 730 may be manufactured using any suitable process. For example, the frame 710 may be a multi-layer component. The interior of the frame 710 may be formed of a more rigid material such as polystyrene by molding, machining, laser-cutting, deposition, and/or 3D printing. One or more layers of coatings that are formed of cell attachment materials suitable for growing and attachment of cells, such as collagen or another suitable protein, may be applied to the surface of the frame 710. The application of the coatings may be applied through immersing the frame 710 to a solution, spraying, and/or deposition. In some cases, not the entire surface area of the frame 710 is applied with coatings. For example, the area of the first cell-growing surface 734 and the area of the second cell-growing surface 736 may be controlled based on the application of coatings. In one embodiment, the ratio of the size of the first cell-growing surface 734 and the size of the second cell-growing surface 736 may be predetermined to control the number of cells to remain on the cell-growing structure 700 in a harvest phase. For example, if the aim is to keep one third of the cells on the cell-growing subunit 730 as seeds for the next cell expansion phase, the total surface area of surfaces that are expected not to be affected by the external force (e.g., the centrifugal force) should constitutes between ten and fifty percent of total surface area of cell-growing surfaces in the cell-growing structure 700.

FIG. 7 also illustrates an expected orientation of the cell-growing structure 700 when a cell-growing container 300 is placed in a centrifuge, in accordance with an embodiment. The centrifuge (not shown in FIG. 7) may be a swing-bucket centrifuge that has a rotational axis 770. During a centrifuge operation, the centrifugal force 760 causes the cell-growing container 300 to turn perpendicular or almost perpendicular to the rotational axis 770, as illustrated in FIG. 7. The cell-growing container 300 and the cell-growing structure 700 may share the same longitudinal axis 702. When the centrifugal force 760 is applied to the cell-growing container 300, a subset of proliferated cells is disassociated from the cell-growing surfaces to the closed end 346 of the cell-growing container 300. The use of mechanical force to disassociate cells, without the introduction of various chemical or acids baths to remove cells, produces more healthy and intact cells. Hence, in some embodiments, an enzyme such as trypsin is not added to disassociate cells.

To make the cell-growing container 300 be re-useable for the next cycle of cell expansion without the need to manually apply initial cells again, another subset of cells should remain on the cell-growing structure 700. To achieve this, the cell-growing structure 700 has a first subset of cell-growing surfaces that are oriented within a range of orientations that is within ten degrees plus or minus of the circular path defined by rotational axis 770 of the centrifuge. In other words, those surfaces are generally tangential (within ten or five degrees plus or minus) to the circular path and are generally orthogonal (within ten or five degrees plus or minus) to the centrifugal force 760 applied by the centrifuge. Hence, those surfaces only experience slight to none centrifugal force 760 so that the cells on those surfaces are not disassociated from the cell-growing structure 700 in a harvest phase. In inset 708, the first cell-growing surface 734 may be an example of those surfaces. In some embodiments, the first subset of surfaces may also be oriented generally orthogonal to the longitudinal axis 702 of the cell growing container 300. in some embodiments, the first subset of surfaces may also be facing opposite to the rotational axis 770 so that any centrifugal force only presses the cells against the frame 710.

The cell-growing structure 700 may also include a second subset of cell-growing surfaces that are arranged outside of the aforementioned range of orientations. The second surface 736 may be an example of the surfaces in the second subset. The second subset of cell growing surfaces experiences significantly stronger centrifugal force so that the cells grown on those surfaces are expected to be disassociated from the surfaces to move to the closed end 346 of the cell-growing container 300. The ratio of the areas of the first subset of surfaces and the second subset of the surfaces may be designed to control the percentage of cells to remain in the cell-growing container 300. For example, for each cycle of cell expansion and harvest, it may be desirable to retain about thirty percent of the cells as the seeds for the next cycle. The total area of the cell-growing surfaces in the first subset (e.g., those surfaces arranged within the range of orientations that is generally tangential to the expected rotational path defined by the rotational axis 770) may constitute between ten and fifty percent of the total cell attachment surface area of the cell-growing structure 700. Other suitable ratios are also possible.

In one embodiment, the centrifuge machine may be a fixed-angle centrifuge. When inserted into the centrifuge and in a centrifuge operation, the longitudinal axis 702 of the cell-growing structure 700 may not be generally perpendicular to the rotation axis. Instead, oftentimes the cell-growing container 300 is inserted at a somewhat diagonal direction and remains at that angle throughout the centrifuge operation. An embodiment of the cell-growing structure 700 may be designed for the fixed-angle centrifuge. For example, the cell-growing structure 700 may have a first subset of surfaces that are arranged generally orthogonal to the centrifugal force of a fixed-angle centrifuge.

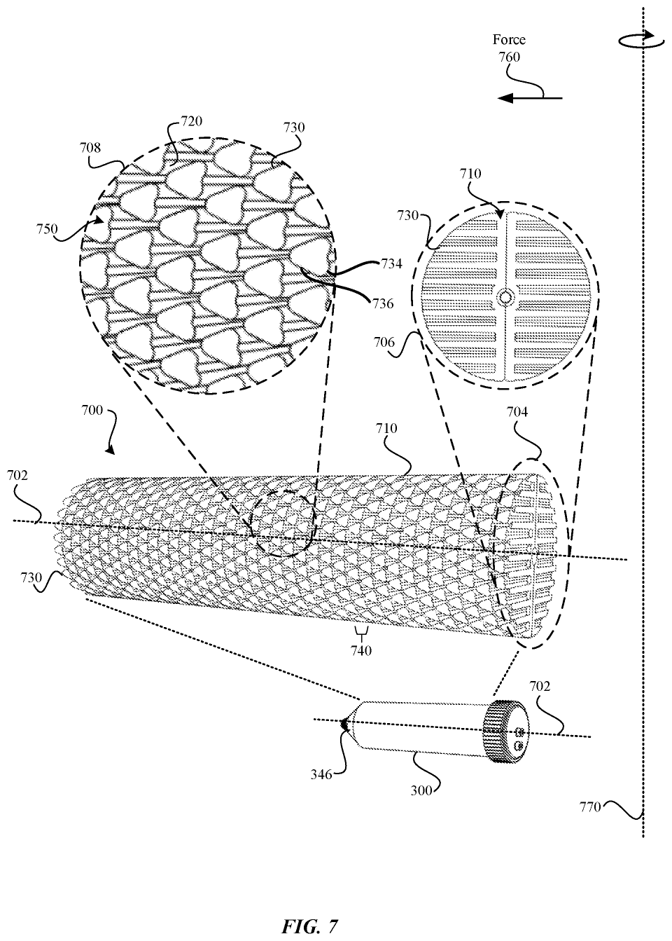

FIG. 8 is a conceptual diagram of different possible cross-section shapes of a cell-growing subunit, in accordance with various embodiments. Each type of cell-growing subunit may have a first surface 802 that is generally orthogonal to the centrifugal force and a second surface 804 that is oriented outside the range of orientations that is generally orthogonal to the centrifugal force. The second surface 804 experience a significant centrifugal force during a centrifuge operation so that cells grown on those surfaces are expected to be disassociated from the cell-growing subunits. The first surfaces 802 are also positioned behind the frame of the cell-growing units to further prevent the cells on those surfaces from disassociating. While centrifuge force is discussed in FIGS. 7 and 8 as an example of external force that can be applied to disassociate cells, other types of forces, such as the gravitational force or sudden acceleration, may also be used to disassociate cells.

### Second Example Cell-Growing Container

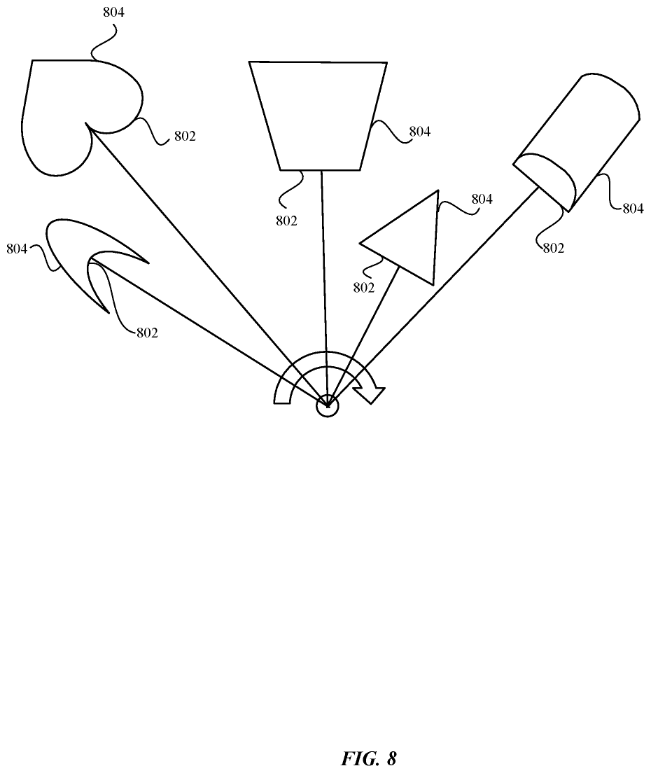

FIG. 9 includes two side views of an example cell-growing container 900, in accordance with an embodiment. The cell-growing container 900 is an example of the cell-growing container 110 that may be used in system 100 shown in FIG. 1. The cell-growing container 900 includes a body 910 that defines a chamber 920 for providing an environment for cell proliferation. The body 910 may include multiple ports, such as a first port 912 and a second port 914, that permit substance exchange between the chamber 920 and another component, such as a pump, a collection container, etc. that is shown in FIG. 1. The body 910 may include a cap 930 and a receptacle 940 that cooperate to define the chamber 920. The cell-growing container 900 may include fewer or additional components that are not shown in various figures. The cell-growing container 900 may also include different components.

The body 910 may include a first surface 942 and a second surface 944 opposite the first surface 942. The body 910 may also include a divider 950 that divides the chamber 920 into a first partition 952 and a second partition 954. The divided partitions may also be referred to as a top partition and a bottom partition. However, the divider 950 may divide the chamber 920 into any other suitable divisions such as left and right, specific quadrants, or other identifiable, symmetrical or not, regular or not, equal size or not, partitions. In one case, the first partition 952 may have a volume that is several times smaller than the second partition 954. The first surface 942 is located in the first partition 952. The first surface 942 may be formed of a material suitable for growing biological cells. In other words, the first surface 942 may be a cell-growing surface.

The second partition 954 may include a plurality of loose cell-growing units 960. The cell-growing units 960 may be loose beads, spheres, ribs, or another other suitable unit having same or different, regular or irregular, shapes and sizes. The cell-growing units 960 have surfaces that are formed of a material suitable for growing biological cells. The cell-growing units 960 may also be referred to as surface-increasing units. The total surface area of the cell-growing units 960 may be at least ten (sometimes hundreds, thousands, tens of thousands) times larger than the first surface 942. Alternatively, or additionally, the second partition 954 may also include a cell-growing structure that is similar to the structure 700 shown in FIG. 7.

The divider 950 may be a porous layer and/or a movable layer that can create a large hole between the first and second partitions 952 and 954. The pores of the divider 950 may be larger than the biological cells so that the cells may migrate from the first partition 952 to the second partition 954. The pores of the divider 950 may be smaller than the cell-growing units 960 to keep the cell-growing units 960 from entering the first partition 952.

The two partitions 952 and 954 allow the cell-growing container 900 to grow a large number of cells by increasing the surface areas provided for growing cells. A small number of initial cells may be manually applied and spread on the first cell-growing surface 942 in the first partition 952 by a user. The application of the cells on the first surface 942 may be similar to a conventional cell culture using a flask. The cells may then be allowed to proliferate for a period of time, often in a closed and pressurized environment as discussed above. After the cells are extensively grown on the first surface 942, a disassociation operation may be performed. For example, an enzyme solution such as trypsin may be added to the chamber 920 through one of the ports 912 or 914. Alternatively, or additionally, an external force such as a centrifugal force or a sudden acceleration may be used to disassociate the cells on the first surface 942. For example, the cell-growing container 900 may be sized and shaped to be insertable to a centrifuge. After the disassociation operation, the enzyme solution may be drained to a collection container. Waste product may also be washed. The released cells are ready for a second phase of growth in the second partition 954. In turn, the cell-growing container 900 is flipped over to a second orientation 970 shown at the bottom of FIG. 9. The loose cells land on the cell-growing units 960 in the second partition 954, which have a significantly larger surface area than the first surface 942 for the cells to expand. In a harvest phase, the cells may be disassociated from the cell-growing units 960 and harvested to a collection container. The cell-growing container 900 may then be flipped over again to the first orientation shown in the top of FIG. 9 for another cycle.

The first surface 942 provides a flat surface for initial cells to adhere to grow to a number large enough to colonize the cell-growing units 960. An initial application of a small number of cells directly to the cell-growing units 960 may sometimes be difficult.

The cell-growing units 960 may be of any sizes. In one embodiment, the cell-growing units 960 may be smaller than at least one of the ports 912 and 914. In one approach of harvesting the cells, the cells may first be disassociated from the surfaces of the cell-growing units 960 and the cells are discharged to a collection container. In another approach, the cell-growing units 960 with the cells still adhered on surfaces may be discharged. A certain number of cell-growing units 960 may remain in the chamber 920 and the cells thereon may serve as seeds in the next cycle. New and sterilized cell-growing units 960 may be replenished to the chamber 920. In yet another approach, the cells on cell-growing units 960 may first be disassociated from surfaces of the cell-growing units 960 and collected in a collection container. The old cell-growing units 960 may, in turn, be replaced with new and sterilized ones to continue other cycles.

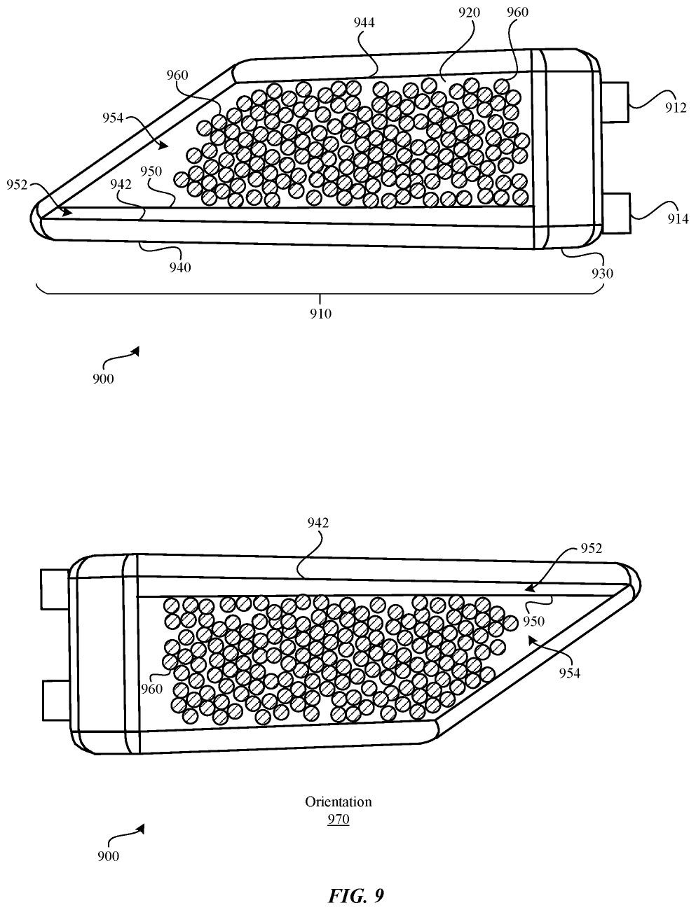

FIG. 10 shows an internal pipette 1000 for the cell-growing container 900, in accordance with an embodiment. The cell-growing container 900 may include one or more internal pipettes 1000. The internal pipette 1000 is movable within the chamber 920. The entire body of the internal pipette 1000 may be rigid or at least some part of the body may be flexible. The internal pipette 1000 may be used to disassociate the cells on a cell-growing surface such as the first surface 942 by mechanically scraping off the cells from the surfaces. For example, at least a rigid part of the internal pipette 1000 may be located in the first partition 952. The rigid part may be connected to a flexible tube that is used to communicate with another component. The pipette 1000 may be controlled by external magnets, levers, gravity, or any other suitable structure or method to control the position of the pipette. The pipette 1000 may include a port 1010 that may be connected to another component to supply or remove materials from the chamber 920. The pipette 1000 may also utilize pressure, internal or external, to add or remove materials from the system. The internal pipette 1000 allows various operations in the chamber 920 while maintaining the cell-growing container 900 in a closed and sterilized environment. The internal pipette 1000 may also be used to harvest cells. For example, the tip of the internal pipette 1000 may be moved around near the cells. Pressure may be applied so that the calls are pushed out of the container through a port that serves as an exit port. Loose cell-growing units 960 (not shown in FIG. 10) may also be discharged in a similar manner.

In one embodiment, the internal moveable pipette 1000 has flexible tubing attached to a post and the other end of the tubing is attached to a port of the cell-growing container 900. An embedded magnet or ferrous material may be attached to the post of the pipette 1000. The magnet or ferrous material may be covered by a thin layer of plastic so the magnet or ferrous material does not come in contact with the cell medium inside the chamber. The position of the pipette 1000 may be controlled from the outside by a handheld magnet through the imbedded magnet or ferrous material.

### Third Example Cell-Growing Container

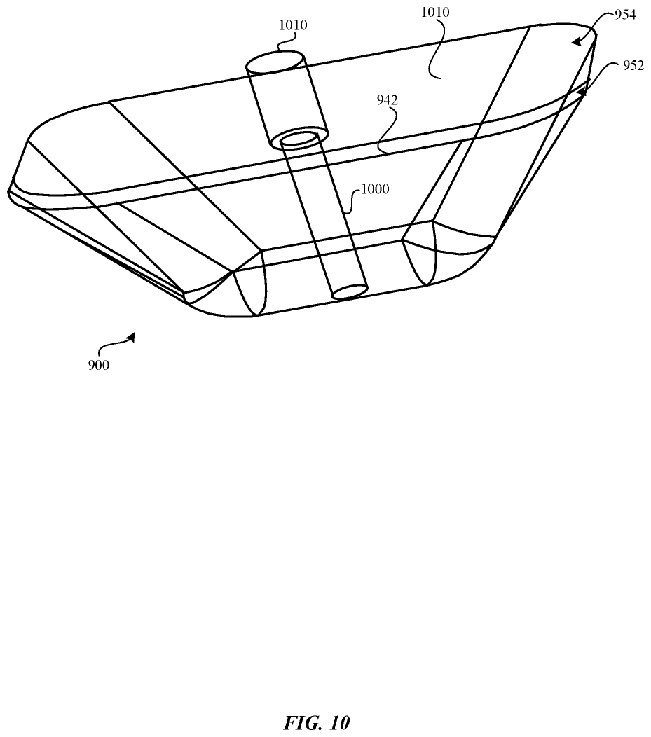

FIG. 11 is a conceptual diagram illustrating an example cell-growing container 1100, in accordance with an embodiment. The cell-growing container 1100 is an example of the cell-growing container 110 that may be used in system 100 shown in FIG. 1. FIG. 11 shows several side views of the cell-growing container 1100, which illustrate different orientations for growing cells. The cell-growing container 1100 includes a body 1110 that defines a chamber 1120 for providing an environment for cell proliferation. The cell-growing container 1100 includes a plurality of cell-growing surfaces, such as the first surface 1130, the second surface 1140, the third surface 1150, and the fourth surface 1160. The cell-growing surfaces 1130, 1140, 1150, and 1160 have different sizes. For example, the first surface 1130 is smaller than the second surface 1140, the second surface 1140 is smaller than the third surface 1150, etc. The increase in the surface area of each surfaces 1130, 1140, 1150, and 1160 may have a predetermined ratio. For example, each larger surface may be a particular number of times (e.g., three times) larger than the next smaller surface. In one embodiment, the first surface 1130 has a surface area of 25 cm², the second surface 1140 has a surface area of 75 cm², the third surface 1150 has a surface area of 225 cm², and the fourth surface 1160 has a surface area of 675 cm². Other ratios are also possible and the increase does not necessarily to be limited to a single fixed ratio. The cell-growing container 1100 may include fewer or more than four cell-growing surfaces and its shape is not limited to having four sides.

The cell-growing container 1100 may be used to progressively expand cells incrementally through increasing surface areas by rotating the cell-growing container 1100 to transfer cells from one surface to another surface that has a larger surface area. For example, a small number of cells may be initially applied to the first surface 1130 and the cell-growing container 1100 is positioned at the orientation 1170. After the cells sufficiently expand on the first surface 1130, a cell disassociation operation may be carried out (e.g., by use of an enzyme and/or an external force) to release the cells from the first surface 1130. The chamber 1120 may be washed. The cell-growing container 1100 may then be flipped and turned to a second orientation 1172. Due to gravity and/or other external force, the released cells fall to the second surface 1140, which is now the bottom surface in the orientation 1172. The process may be repeated for the third orientation 1174 and the fourth orientation 1176. In some cases, the entire process may be conducted in a closed and sterilized manner. The cell-growing container 1100 may include ports (not shown) that are used to communicate with other components (e.g., a pump, a substance source). By using the port, after cells proliferate on the largest surface, loose cell-growing units such as beads may be added to the chamber 1120 through the port. The proliferated cells on the surface may be dropped to the cell-growing units to continue another phase of cell expansion with additional surface areas.

### Fourth Example Cell-Growing Container

FIGS. 8A and 8B illustrate another example cell-growing container 1200, in accordance with an embodiment. FIG. 8A is a perspective view of the cell-growing container 1200. FIG. 8B shows the cross-section of the cell-growing container 1200. The cell-growing container 1200 includes a body 1210 that defines a chamber 1220 for carrying cells and providing an environment for cell proliferation. The body 1210 may include multiple ports, such as two input ports 1212 and two output ports 1214, that permit substance exchange between the chamber 1220 and another component, such as a pump, a collection container, etc. that is shown in FIG. 1. The body 1210 may include a cap 1230 and a receptacle 1240. The body 1210 may carry a cell-growing structure 1250 that may serve as the substrate for growing a large number of cells. The cell-growing container 1200 may include fewer or additional components that are not shown in various figures. The cell-growing container 1200 may also include different components.

The cell-growing container 1200 includes a motor shaft 1260 that can be attached to a motor (not shown in the figures). The cell-growing container 1200 may be sized and shaped to be insertable into a rotating device such as a blender that has a motor. The motor shaft 1260 may protrude from the body 1210 and attached to the cell-growing structure 1250. Hence, the cell-growing structure 1250 may be driven by a motor and is rotatable relative to the body 1210 when the chamber 1220 of the cell-growing container 1200 remained closed from an external environment.

The cell-growing structure 1250 is another example of a reticulated structure that is a surface-increasing structure. The cell-growing structure 1250 includes a plurality of cell-growing units 1270 that may be fanned radially and spaced apart to form a plurality of spaces. The cell-growing units 1270 may also be arranged in layers. Two example layers are shown in FIGS. 8A and 8B. An example shape of cell-growing unit 1270 is shown at the right side of the cell-growing container 1200 in FIG. 12A. A cell-growing unit 1270 may include a plurality of cell-growing surfaces. For example, a first surface 1272 (e.g. a top surface) and a second surface 1274 (e.g. a bottom surface) may be generally orthogonal (within 5 or 10 degrees of perpendicularity) to a longitudinal axis defined by the motor shaft 1260. The cell-growing unit 1270 may also include two side surfaces 1276, an inner radial surface 1278, and an outer radial surface 1280. In a disassociation operation that is driven by a motor connected to the motor shaft 1260, cells on the inner radial surface 1278 may remain on the surface because the surface 1278 is generally tangential to the circular path defined by the rotation and the surface 1278 also work against the centrifugal force to help the cells to stay on the surface 1278. Cells on other surfaces may be disassociated from the surfaces.

The cell-growing container 1200 may also be inserted into another type of force-generating machine other than a motor. For example, the cell-growing container 1200 may be connected to a vertical shaker that has an actuator to accelerate and decelerate the cell-growing container 1200 in a direction along the longitudinal axis defined by the motor shaft 1260 (e.g., up and down motion). Cells from vertical surfaces such as surfaces 1276, 1278, and 1280 are disassociated from the surfaces while cells from horizontal surfaces 1272 and 1274 (surfaces that are generally orthogonal to the direction of the force) remain on the surfaces.

Although in this disclosure the cell-growing container 110, cell-growing container 300, the cell-growing container 900, the cell-growing container 1100, and the cell-growing container 1200 each include some unique features, the features described in one cell-growing container may also be presented in another cell-growing container even though this disclosure does not expressly mention the presence of the feature. For example, each cell-growing container may include one or more ports for communication with another component, even though the ports are not shown. Also, the cell-growing container 300 may include the internal pipette 1000 shown in FIG. 10. Likewise, the individual cell-growing units 960 such as loose beads or the cell-growing structure 350 may be presented in the chamber 1120 of the cell-growing container 1100.

### Example Cell-Growing Process

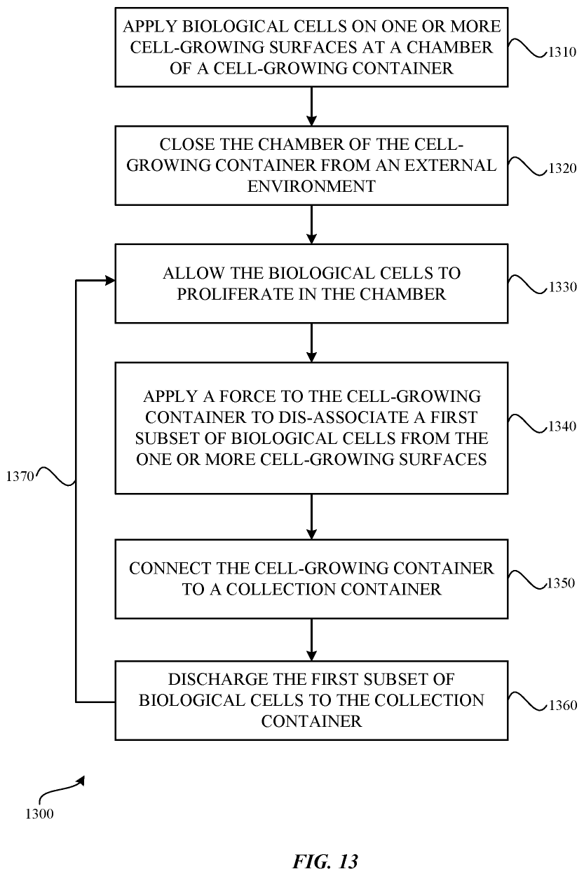

FIG. 13 is a flowchart depicting an example cell-growing process 1300, in accordance with an embodiment. A user may use a cell-growing system, such as the system 100, to perform the process 1300. In one embodiment, the user applies 1310 biological cells on one or more cell-growing surfaces at a chamber of a cell-growing container. The cell-growing surfaces may be one or more of the surfaces of the cell-growing structure 350 of the cell-growing container 300, the first surface 942 of the cell-growing container 900, or the first surface 1130 of the cell-growing container 1100. The application of the cells may be conducted by any suitable method, such as using a sterilized spreader or using an internal pipette 1000.

In one embodiment, the user closes 1320 the chamber of the cell-growing container from an external environment. For example, the cell-growing container with the initial cells may be connected to a pump and one or more substance sources. The entire system may be closed relative to the external environment. In one embodiment, a filter may be used to sterilize and keep containments and other unwanted substances from entering the chamber of the cell-growing container. Additionally, or alternatively, the chamber may be pressurized to reduce the chance of foreign unwanted objects entering the chamber. For example, the pressure of the chamber may be regulated through a pump that is connected to the cell-growing container. The pump may maintain the pressure of the chamber to a value that is higher than the atmospheric pressure.

In one embodiment, the user allows 1330 the biological cells to proliferate in the chamber in a cell expansion phase in a closed and sterilized environment. During a cell expansion phase, both the pressure and substance supplied to the cell-growing container may be regulated. For example, using a flow control regulator connected to the cell-growing container, the amount of oxygen and carbon dioxide in the chamber may be regulated to mimic the native biological environment of the biological cells. Other substances such as nutrients supplied to the chamber may also be regulated through one or more flow control regulator. The cells may be allowed to proliferate in a sterilized and closed environment for a period of time such as hours or days. In some embodiments, the cells may proliferate in a temperature-controlled environment to simulate the body temperature. For example, the cell-growing container may be immersed in an incubator. Occasionally cell samples or even a cell-growing unit may be removed from the chamber for testing purpose. For some biological cells, the chamber may be completely sealed during cell expansion without connecting to any substance source.

In a harvest phase, the user applies 1340 a force to disassociate a first subset of biological cells from the cell-growing surfaces. A second subset of biological cells may remain on the cell-growing surfaces. The external force may be a centrifugal force, the gravitational force, or a force in association with sudden acceleration such as a force that is generated by a mechanical shaker. For example, the user may detach the cell-growing container from other components (e.g., the pump) and insert the cell-growing container to a centrifuge. A centrifugal force is applied to the cell-growing container through the centrifuge. The level of centrifugal force and the duration of the centrifuge operation may be controlled to regulate the number of cells that get disassociated by the force. Some of the cell-growing surfaces in, for example, the cell-growing structure 350 may not experience as a strong centrifugal force as other cell-growing surfaces. As a result, some cells are disassociate from the cell-growing surfaces while others remain. In another example, applying an external force may include re-orientating the cell-growing container to apply the gravitational force to disassociate some of the cells from the cell-growing surfaces. In yet another example, applying an external force may include applying an acceleration to the cell-growing container. The cell-growing container may be attached to a mechanical shaker to accelerate and decelerate the container. In addition to or alternative to applying an external force, the user may also add an agent such as a disassociation enzyme to the cell culture to release the cells. In some cases, only mechanical force is used to disassociate the cells. No chemical or biological agent is added to avoid damaging the cells.

Washing may also be performed before cells are harvested to a collection container. For example, fluid is pumped into the chamber of the cell-growing container through a port that has an inline sterile filter or sterile connection. The fluid is passed through the chamber of the cell-growing container in a sterile fashion to wash the cells so as to remove waste products and residual enzyme such as trypsin. Washing is to be done carefully so that excess trypsin and or waste does not get into and/or on other areas of the chamber that are desired to be used during another expansion. The waste fluid exits the chamber through another port. Because the chamber may be pressurized, contaminates cannot get in through the second port.

In one embodiment, the user connects 1350 the cell-growing container to a collection container to harvest the cells. During the harvest phase, the cell-growing container remains closed from the external environment when it is connected to the collection container. For example, the collection container may be connected to the cell-growing container through a port of the cell-growing container that has a switch to maintain the closed environment of the chamber. The first subset of cells, which are the cells that are disassociated, is discharged 1360 to the collection container. For example, a pump may be used to push the cells to the collection container. In some embodiments in which loose cell-growing units are used in the cell-growing container, the cell-growing units may also be discharged. New and sterilized cell-growing units may be replenished.

The cell-growing system may also be used as a system for growing suspension cells. In growing suspension cells, the internal cell-growing structure located in the chamber may be removed. The chamber may be filled with the cell medium solution. In a harvest phase, a portion of the cell medium, along with the proliferated cells, may be drained to a collection container. In one case, a U-shape tube may be presented inside the chamber to preserve a small amount of the cell medium. After cells are harvested, new and sterilized cell medium solution may be added to the chamber for the next cell expansion cycle.

After the harvest, the cell-growing system may undergo a new cycle of cell expansion and harvest, as indicated by the arrow 1370. For example, the user may allow the second subset of biological cells, which are the cells that remain on the cell-growing surfaces, to act as seeds for proliferation in the closed chamber after the first subset of biological cells is discharged to the collection container. The process may be repeated multiple times to generate a large number of cells. Throughout the process, the chamber of the cell-growing container may remain sterilized and closed from the external environment. The repeat of cycles allows the system to be re-used without a manual re-application of cells. In other words, in some embodiment, the cells only need to be manually applied once initially.

### Additional Considerations

Beneficially, a closed system can address the expansion of adherent cells, suspension cells, or both in combination within the same closed system. The devices can drastically reduce the need for laminar flow hoods and clean rooms to expand and maintain living cells. The devices can also be sized to expand cells to small quantities or to quantities in the billions while maintaining sterility. In one embodiment, the system can expand cells in a cell-growing structure that has 64 of 225 cm² surfaces, with a total of 14,400 cm² of surface, to produce approximate 1.5 billion cells. Also, the cell-growing structure described herein allows the disassociation of cells from a substrate using a mechanical force without the use of chemicals or enzyme that may damage the cells. When an enzyme such as trypsin is used to disassociate cells, the surfaces of the cells may be damaged and the cells may not repopulate a cell-growing surface. Also, the enzyme is often a protein dissolver that might destroy other proteins and nutrients. By using mechanical force to disassociate cells from cell-growing surfaces, the proteins and nutrients left behind by the cells will help the proliferation of cells in the next cycle of cell expansion. In some cases, the cells could expand in the next cycle twice as fast as they are initially grown on a surface.

The systems described herein are especially suitable for growing stem cells because the tightly controlled and sterilized environment reduces the chances of the stem cells starting to differentiate due to environmental change. By maintaining sterility when adding or removing components and cells from the system, cells can be taken for testing or harvested in a large batch from the system at any time without compromising sterility.

The foregoing description of the embodiments has been presented for the purpose of illustration; it is not intended to be exhaustive or to limit the patent rights to the precise forms disclosed. While particular embodiments and applications have been illustrated and described, it is to be understood that the invention is not limited to the precise construction and components disclosed herein and that various modifications, changes and variations which will be apparent to those skilled in the art may be made in the arrangement, operation and details of the method and apparatus disclosed herein without departing from the spirit and scope of the present disclosure. Persons skilled in the relevant art can appreciate that many modifications and variations are possible in light of the above disclosure.

Any feature mentioned in one claim category, e.g. method, can be claimed in another claim category, e.g. computer program product, system, device, as well. The dependencies or references back in the attached claims are chosen for formal reasons only. Any subject matter resulting from a deliberate reference back to any previous claims (in particular multiple dependencies) can be claimed as well, so that any combination of claims and the features thereof is disclosed and can be claimed regardless of the dependencies chosen in the attached claims. The subject-matter which can be claimed comprises not only the combinations of features as set out in the disclosed embodiments but also any other combination of features from different embodiments. Various features mentioned in the different embodiments can be combined with explicit mentioning of such combination or arrangement in an example embodiment. Furthermore, any of the embodiments and features described or depicted herein can be claimed in a separate claim and/or in any combination with any embodiment or feature described or depicted herein or with any of the features.

The term “steps” does not mandate or imply a particular order. For example, while this disclosure may describe a process that includes multiple steps sequentially with arrows present in a flowchart, the steps in the process do not need to be performed by the specific order claimed or described in the disclosure. Some steps may be performed before others even though the other steps are claimed or described first in this disclosure. Likewise, any use of (i), (ii), (iii), etc., or (a), (b), (c), etc. in the specification or in the claims, unless specified, is used to better enumerate items or steps and also does not mandate a particular order.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein. In addition, the term “each” used in the specification and claims does not imply that every or all elements in a group need to fit the description associated with the term “each.” For example, “each member is associated with element A” does not imply that all members are associated with an element A. Instead, the term “each” only implies that a member (of some of the members), in a singular form, is associated with an element A. In claims, the use of a singular form of a noun may imply at least one element even though a plural form is not used.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights.

What is claimed is:

1. A cell-growing container for growing mammalian cells, the cell-growing container comprising:
a body defining a chamber;
a reticulated structure located in the chamber, the reticulated structure comprising a frame, the frame defining a volume of the reticulated structure and creating a plurality of spaces on and inside the volume, the frame comprising a plurality of cell-growing subunits, at least one of the cell-growing subunits comprising a first surface formed of a cell attachment material suitable for attachment of mammalian cells and a second surface formed of the cell attachment material and oriented differently from the first surface; and
a first port through the body, the first port configured to permit substance exchange between the chamber and an external source, the first port being sealable.

2. The cell-growing container of claim 1, wherein the body is sized and shaped to be insertable into a centrifuge and be held by the centrifuge in a centrifuge operation.

3. The cell-growing container of claim 2, wherein the first surface is generally orthogonal to a direction of a centrifugal force generated by the centrifuge during the centrifuge operation, the first surface within a range of orientations of plus or minus ten degrees.

4. The cell-growing container of claim 3, wherein the second surface is arranged outside of the first surface range of orientations.

5. The cell-growing container of claim 3, wherein the total area of cell-growing surfaces of the reticulated structure arranged within the range of orientations constitutes between ten and fifty percent of the total cell attachment surface area of the reticulated structure.

6. The cell-growing container of claim 1, wherein the body comprises a receptacle and a cap that is removably engaged with the receptacle to form the chamber.

7. The cell-growing container of claim 1, wherein the at least one of the cell-growing subunits comprises coatings on the first and second surfaces, the coatings formed of the cell attachment material.

8. The cell-growing container of claim 1, wherein the material is hydrophilic.

9. The cell-growing container of claim 1, wherein the material includes at least collagen.

10. The cell-growing container of claim 1, further comprising:
a second port at the body, the second port configured to be connected to a collection container, and the first port configured to be connected to a pump.

11. The cell-growing container of claim 1, wherein the volume of the reticulated structure is at least fifty percent of the chamber's volume.

12. The cell-growing container of claim 1, wherein the reticulated structure is removable from the body.

13. The cell-growing container of claim 1, wherein the total surface area of the reticulated structure that is formed of the material is at least ten times larger than the inner surface area of the body.

14. A system for growing mammalian cells in a closed environment, the system comprising:
a cell-growing container for growing mammalian cells, the cell-growing container comprising a body and a first port, the body defining a chamber;
a pump in communication with the cell-growing container through the first port, the pump configured to regulate pressure of the chamber; and
a filter located between the cell-growing container and the pump, the filter configured to prevent contamination of the chamber.

15. The system of claim 14, further comprising:
a flow control regulator in communication with the first port, the flow control regulator configured to be connected to one or more sources of substances and configured to regulate amounts of substances supplied to the chamber.

16. The system of claim 15, wherein the one or more sources of substances comprise an oxygen source and a carbon dioxide source.

17. The system of claim 14, wherein the cell-growing container further comprises a second port, and the system further comprises a collection container in communication with the second port, the collection container configured to receive substances discharged from the cell-growing container.

18. The system of claim 14, wherein the cell-growing container further comprises a reticulated structure located in the chamber, the reticulated structure comprising a frame, the frame defining a volume of the reticulated structure and creating a plurality of spaces on and inside the volume, the frame comprising a plurality of cell-growing subunits, at least one of the cell-growing subunits comprising a first surface formed of a cell attachment material suitable for attachment of mammalian cells and a second surface formed of the cell attachment material and oriented differently from the first surface.

19. The system of claim 14, wherein the body of the cell-growing container is sized and shaped to be insertable into a centrifuge and be held by the centrifuge in a centrifuge operation.

20. The system of claim 14, wherein the cell-growing container further comprises:
a divider dividing the chamber into at least a first partition and a second partition;
a cell-growing surface within the first partition, the cell-growing surface formed of a material suitable for growing mammalian cells; and
a plurality of cell-growing units carried within the second partition, the cell-growing units having surfaces that are formed of the material.

21. The system of claim 20, wherein the cell-growing container further comprises an internal pipette that is movable within the chamber.

22. The system of claim 20, wherein the total surface area of the plurality of cell-growing units is at least ten times larger than the inner surface area of the chamber.

23. The system of claim 14, wherein the cell-growing container further comprises a plurality of cell-growing surfaces, at least a first cell-growing surface being smaller than a second cell-growing surface, the second cell-growing surface being smaller than a third cell-growing surface.

24. The system of claim 14, wherein the cell-growing container further comprises:
a motor shaft that is capable of being connected to a motor,
a cell-growing structure attached to the motor shaft, the cell-growing structure located inside the chamber of the cell-growing container, the cell-growing structure rotatable relative to the body of the cell-growing container.

25. A reticulated structure for growing mammalian cells, the reticulated configured to be insertable into a cell-growing container that is sealable, the reticulated structure comprising:
a frame defining a volume of the reticulated structure and creating a plurality of spaces on and inside the volume, the frame comprising a plurality of cell-growing subunits, at least one of the cell-growing subunits comprising a first surface formed of a cell attachment material suitable for attachment of mammalian cells and a second surface formed of the cell attachment material and oriented differently from the first surface.

26. A method comprising:
applying mammalian cells on one or more cell-growing surfaces of a cell-growing container;
closing the cell-growing container to an external environment;
allowing the mammalian cells to proliferate in the cell-growing container;
applying a force external to the cell-growing container to disassociate a first subset of mammalian cells from the one or more cell-growing surfaces, a second subset of mammalian cells remaining on the one or more cell-growing surfaces;
connecting the cell-growing container to a collection container, the cell-growing container remained closed to the external environment when connected to the collection container; and
discharging the first subset of mammalian cells to the collection container.

27. The method of claim 26, further comprising:
allowing the second subset of mammalian cells to proliferate in the closed cell-growing container after the first subset of mammalian cells is discharged.

28. The method of claim 26, further comprising:
regulating pressure of the cell-growing container through a pump that is connected to the cell-growing container.

29. The method of claim 28, wherein regulating the pressure of the cell-growing container comprises maintaining the pressure of the cell-growing container to a value that is higher than atmospheric pressure.

30. The method of claim 26, further comprising:
regulating substances supplied to the cell-growing container through a flow control regulator connected to the cell-growing container.

31. The method of claim 30, wherein regulating substances supplied to the cell-growing container comprises regulating an amount of oxygen and carbon dioxide in the cell-growing container to mimic a native biological environment of the mammalian cells.

32. The method of claim 26, wherein the cell-growing container comprises a reticulated structure carried in a chamber of the cell-growing container, the reticulated structure comprising a frame, the frame defining a volume of the reticulated structure and creating a plurality of spaces on and inside the volume, the frame comprising a plurality of cell-growing subunits, at least one of the cell-growing subunits comprising a first surface formed of a cell attachment material suitable for attachment of mammalian cells and a second surface formed of the cell attachment material and oriented differently from the first surface; and
wherein the first surface and the second surface being part of the one or more cell-growing surfaces.

33. The method of claim 26, wherein applying a force external to the cell-growing container to disassociate the first subset of mammalian cells from the one or more cell-growing surfaces comprises:
inserting the cell-growing container to a centrifuge; and
applying a centrifugal force to the cell-growing container through the centrifuge, the centrifugal force being the force external to the cell-growing container.

34. The method of claim 26, wherein applying a force external to the cell-growing container to disassociate the first subset of mammalian cells from the one or more cell-growing surfaces comprises:
re-orienting the cell-growing container to apply a gravitational force to disassociate the first subset of mammalian cells from the one or more cell-growing surfaces, the gravitational force being the force external to the cell-growing container.

35. The method of claim 26, wherein applying a force external to the cell-growing container to disassociate the first subset of mammalian cells from the one or more cell-growing surfaces comprises:
applying an acceleration to the cell-growing container, a force causing the acceleration being the force external to the cell-growing container.

36. The method of claim 25, wherein the force is initiated by a mechanical shaker.
